(19) United States
(12) Patent Application Publication
(10) Pub. No.: US 2022/0403448 A1
(43) Pub. Date: Dec. 22, 2022

(54) PRIMER, PROBE AND KIT FOR TUBERCULOSIS DIAGNOSIS USING URINE SAMPLE, AND USES THEREOF

(71) Applicant: REPUBLIC OF KOREA(MASAN NATIONAL TUBERCULOSIS HOSPITAL), Gyeongsangnam-do (KR)

(72) Inventor: Sungweon RYOO, Gyeongsangnam-do (KR)

(21) Appl. No.: 17/776,315

(22) PCT Filed: Nov. 10, 2020
(86) PCT No.: PCT/KR2020/015712

§ 371 Date: May 12, 2022

(30) Foreign Application Priority Data
Nov. 19, 2019 (KR) 10-2019-0148294

(51) Int. Cl. C12Q 1/689; C12Q 1/6818

(52) U.S. Cl. CPC C12Q 1/689; C12Q 1/6818

(57) ABSTRACT

Disclosed are a primer and a probe for detecting M. tuberculosis trDNA in urine, a kit comprising the same, an information providing method for tuberculosis diagnosis, and a tuberculosis treatment monitoring method using the same. As the primer and probe of the present invention have high sensitivity, they can accurately detect even a very low concentration of M. tuberculosis trDNA present in a urine sample, thereby providing information to allow an accurate diagnosis in the early stage of tuberculosis infection and having great utility in checking for drug resistance of the M. tuberculosis and treatment efficacy by monitoring drug treatment of tuberculosis. Further, the primer, probe, and kit and the methods using the same of the present invention are based on trDNA present in urine and thus are non-invasive, thereby providing convenience for patients and having great utility.

### TECHNICAL FIELD

The present invention relates to a primer, a probe, and a kit for tuberculosis diagnosis using a urine sample, and more particularly, to a primer and a probe for detecting Mycobacterium tuberculosis trDNA in urine, a kit comprising the same, and an information providing method for tuberculosis diagnosis and a tuberculosis treatment monitoring method using the same.

### BACKGROUND ART

Globally, tuberculosis is an important disease that affects new 8.7 million patients and kills 1.4 million people each year. In particular, Korea has the highest incidence of tuberculosis per unit population among OECD countries, with an incidence of tuberculosis of 80 per 100,000 population. Early diagnosis and treatment of tuberculosis are very important nationally (Korean Patent No, 10-1568736).

Although there are various methods, such as clinical imaging, chest X-rays, acid-fast bacillus smear tests, M. tuberculosis nucleic acid amplification tests, and acid-fast bacillus culture tests to diagnose tuberculosis, M. tuberculosis need to be detected in a culture test to confirm tuberculosis. However, in the case of a smear test, sensitivity is as low as 25% to 80%, and in the case of a culture test, it takes 2 to 4 weeks, so it is often difficult to confirm tuberculosis in actual clinical practice.

Whether the smear test is negative, whether M. tuberculosis detected in a culture test, whether chest radiographs are improved, and the like are used as methods for monitoring the treatment of tuberculosis, but there are cases where treatment monitoring is not easy in various clinical situations, such as when the smear test is negative from the beginning and when the improvement in chest radiographs is ambiguous.

In treating tuberculosis, rapid identification of multidrug-resistant tuberculosis patients is also an important part. To this end, a rapid resistance test is performed. A drug susceptibility test is performed based on a bacterial culture result, but not all multidrug-resistant tuberculosis strains can be found in the case of the rapid resistance test. M. tuberculosis culture, it takes an additional 2 to 4 weeks. Therefore, if the treatment response can be quantitatively monitored in the early stage of tuberculosis treatment when the quantitative value does not decrease, it may be helpful in the rapid discovery of multidrug-resistant strains.

Tr-DNA (Transrenal DNA; trDNA) is a relatively recently discovered class of extracellular urinary DNA derived from dying cells throughout the body. Although post-apoptotic DNA is known to be observed in circulating plasma, some of the DNA fragments cross the kidney barrier and are detected in urine in the form of fragments with a size of 90 to 200 bp.

TrDNA, including a fetal base sequence, has been isolated from the urine of pregnant women, tumor-specific mutations have been found in the urine of several cancer patients, including colon and pancreatic cancer patients, and trDNA has been found in the urine of a recipient who has been transplanted with the DNA of an organ transplant donor. In addition, proviral HIV DNA, bacterial and parasitic DNA, and the like have been detected as trDNA in the urine of patients infected with them. Therefore, the potential application range of trDNA-based analysis is expected to be widely used in the fields of molecular diagnosis and genetic testing, such as fetal genetic disease testing, tumor testing, treatment progress monitoring, and infectious material detection.

### SUMMARY

### Technical Problem

Thus, the present inventors have continuously researched to meet the needs of the prior art, and as a result, identified the presence of a new trDNA of M. tuberculosis in urine, and identified that when primers and probes capable of detecting the trDNA were designed and used to conduct a polymerase chain reaction (PCR), surprisingly, even a minimal amount of the trDNA could be detected so that tuberculosis could be diagnosed quickly and accurately with high specificity and sensitivity and convenience for patients is also high using a urine sample, thereby completing the present invention.

Therefore, the object of the present invention is to provide a primer for detecting M. tuberculosis trDNA in urine.

Another object of the present invention is to provide a probe for detecting M. tuberculosis trDNA in urine.

Still another object of the present invention is to provide a kit for detecting M. tuberculosis trDNA in urine, including the primer or probe.

Yet another object of the present invention is to provide an information-providing method for tuberculosis diagnosis using the primer, probe, or kit.

Yet another object of the present invention is to provide a tuberculosis treatment monitoring method using the primer, probe, or kit.

### Technical Solution

The present invention provides a primer for detecting M. tuberculosis trDNA in urine to achieve the objects.

In the present invention, the M. tuberculosis trDNA is found in the urine of tuberculosis patients and is a 99 to 200 bp DNA including the 99 bp base sequence (SEQ ID NO: 2) from the 2075^{th} base to the 2173^{rd} base in an IS6110 gene base sequence (SEQ ID NO: 1) of M. tuberculosis.

The present inventors first identified that the M. tuberculosis trDNA is present in the urine of tuberculosis patients.

The term ‘primer’ refers to an oligonucleotide of a single-stranded base sequence identical to or complementary to one nucleic acid strand in double-stranded DNA to be replicated. It means a short nucleic acid sequence that can serve as a starting point for synthesizing a primer extension product. DNA synthesis can be initiated in the presence of a reagent for polymerization (DNA polymerase or reverse transcriptase) and four different deoxynucleotide triphosphates (dNTPs) in a proper buffer and at a proper temperature.

In the present invention, the primer refers to a set of forward and reverse primers, including a base sequence identical to or complementary to a part of the base sequence of M. tuberculosis trDNA of SEQ ID NO: 2.

In the present invention, the primer includes a base sequence of 10 to 25 bp, more preferably, a base sequence of 14 to 20 bp. Most preferably, the primer in the present invention is a set of a forward primer with the base sequence of SEQ ID NO: 3 and a reverse primer with the base sequence of SEQ ID NO: 4.

The primer set of the forward primer having the base sequence of SEQ ID NO: 3 and the reverse primer, which is the base sequence of SEQ ID NO: 4, was synthesized based on the base sequence (SEQ ID NO: 2) of the M. tuberculosis trDNA found by the present inventors (FIG. 1), and the M. tuberculosis trDNA can be detected with high sensitivity in urine samples by using the primer set (FIGS. 7, 8A and 8B).

According to another object of the present invention, the present invention provides a probe for detecting M. tuberculosis trDNA in urine.

In the present invention, the probe includes a base sequence identical to or complementary to a part of the base sequence of M. tuberculosis trDNA of SEQ ID NO: 2.

The probe includes a base sequence of 10 to 25 bp, more preferably, a base sequence of 14 to 20 bp. Most preferably, the probe in the present invention has the base sequence of SEQ ID NO: 5.

The probe having the base sequence of SEQ ID NO: 5 was synthesized based on the base sequence (SEQ ID NO: 2) of the M. tuberculosis trDNA found by the present inventors (FIG. 1), and the M. tuberculosis trDNA can also be detected with high sensitivity in urine samples by using the probe (FIGS. 7, 8A and 8B).

In the present invention, the 5′ and 3′ ends of the probe are preferably labeled with a fluorescent material, the 5′ end-labeled fluorescent material is a reporter selected from the group consisting of 6-carboxyfluorescein (FAM), hexachloro-6-carboxyfluorescein (HEX), tetrachloro-6-carboxyfluorescein, and Cyanine-5 (Cy5), and the 3′ end-labeled fluorescent material is preferably a quencher which is 6-carboxytetramethyl-rhodamine (TAMRA) or black hole quencher-1,2,3 (BHQ-1,2,3), but is not limited thereto.

When the reporter and the quencher are present in close proximity to both ends of the probe, the fluorescence of the reporter is not detected by canceling the fluorescence. Still, as the amplification progresses, the fluorescence of the reporter is sensed when the reporter moves away from the quencher. Therefore, the intensity of fluorescence gradually increases as the amplification cycle increases.

According to the present invention, the primer and probe can detect the M. tuberculosis trDNA through PCR (polymerase chain reaction). The type of PCR is not limited, and real-time PCR and droplet digital PCR are preferable because the reaction result can be monitored in real-time by using primers and/or probes.

According to another object of the present invention, provided is a kit for detecting M. tuberculosis trDNA in urine, including the primer or probe.

In the present invention, the kit may additionally include an amplification buffer, dNTPs, a control, a detection reagent, an instruction manual, and the like, in addition to the primer or probe of the present invention. In addition, the kit may be provided in a liquid or dry type and may include additional components depending on the purpose as long as there is no effect on the reaction.

According to still another object of the present invention, the present invention provides an information providing method for tuberculosis diagnosis, the method including:

i) extracting M. tuberculosis trDNA including the base sequence of SEQ ID NO: 2 from a urine sample of a subject;

ii) amplifying, by PCR, the M. tuberculosis trDNA as a template using the primer set or the probe; and

iii) determining the presence or absence of infection with Mycobacterium tuberculosis by examining the presence or absence of a product by the amplification or the fluorescence value of the product.

In the present invention, the ‘subject’ means a tuberculosis patient or a person suspected of having tuberculosis.

In the present invention, M. tuberculosis trDNA may be extracted according to a typical DNA extraction method.

The method of the present invention has high sensitivity. It can detect M. tuberculosis DNA even when a minimal amount of M. tuberculosis trDNA is present in a urine sample. In particular, in the case of a real-time polymerase chain reaction and a droplet digital chain polymerization reaction, it is possible to observe whether or not the amplification is immediately performed while the amplification is progressing. The information provision time for tuberculosis diagnosis can be reduced because a separate amplification product confirmation step is not required.

Furthermore, according to yet another object of the present invention, the present invention provides a method of monitoring drug treatment of tuberculosis; the method includes:

i) extracting M. tuberculosis trDNA including the base sequence of SEQ ID NO: 2 from a urine sample of a tuberculosis patient;

ii) amplifying, by PCR, the M. tuberculosis trDNA as a template using the primer set or the probe; and

iii) determining the presence or absence of a response to drug treatment by examining the presence or absence of a product by the amplification or the fluorescence value of the product.

The drug refers to a drug used to treat tuberculosis.

Since the present invention method has high sensitivity, even a very small amount of M. tuberculosis trDNA present in a urine sample can be detected, so it is possible to monitor the treatment of tuberculosis, such as drug treatment progress or multidrug resistance of tuberculosis patients.

### Advantageous Effects

According to the primer, probe, and kit, and the methods using the same according to the present invention, it is possible to quickly and conveniently diagnose M. tuberculosis in a urine sample. In addition, the primer and probe, according to the present invention, have high sensitivity and can accurately detect even a very low concentration of M. tuberculosis trDNA present in a urine sample, thereby providing information to allow an accurate diagnosis in the early stage of tuberculosis infection and having great utility in checking for drug resistance of the M. tuberculosis and treatment efficacy by monitoring drug treatment of tuberculosis.

Further, the primer, probe, and kit and the methods using the same of the present invention are based on trDNA present in urine and thus are non-invasive, thereby providing convenience for patients and having great utility.

### BRIEF DESCRIPTION OF THE DRAWINGS

FIG. 1 illustrates the IS6110 gene base sequence of M. tuberculosis and the position of the 99 bp trDNA from the 2075^{th} base to the 2173^{rd} base among it.

FIG. 2 is a schematic view illustrating the positions of primers and a probe in M. tuberculosis trDNA according to the present invention.

FIG. 3A is a graph showing real-time PCR results of template DNA using the primer and probe according to the present invention and

FIG. 3B is a graph showing average PCR cycle values in which template DNA is detected at each concentration.

FIG. 4A is a graph showing the results of droplet digital PCR of template DNA using the primer and probe according to the present invention. Based on the Threshold Value (450) line, the upper dots represent positive droplets, and the lower dots represent negative droplets.

FIG. 4B is a graph showing the average value of the copy numbers according to the template DNA at each concentration.

FIG. 5A is a graph showing the results of real-time PCR of DNA extracted from H37Rv using the primer and probe according to the present invention and

FIG. 5B is a graph showing the average PCR cycle value in which H37Rv DNA is detected at each concentration.

FIG. 6A is a graph showing the results of droplet digital PCR of H37Rv DNA using the primer and probe according to the present invention. Based on the Threshold Value (457) line, the upper dots represent positive droplets, and the lower dots represent negative droplets.

FIG. 6B is a graph showing the average value of the copy numbers according to the H37Rv DNA at each concentration.

FIG. 7 is a graph showing the PCR cycle average value in which M. tuberculosis trDNA is detected at each concentration as a result of real-time PCR of M. tuberculosis trDNA extracted from the urine of tuberculosis patients using the primer and probe according to the present invention.

FIG. 8A is a graph showing the results of droplet digital PCR of M. tuberculosis trDNA extracted from the urine of tuberculosis patients using the primer and probe according to the present invention. Based on the Threshold Value (457) line, the upper dots represent positive droplets, and the lower dots represent negative droplets.

FIG. 8B is a graph showing the average value of the copy numbers according to M. tuberculosis trDNA extracted from the urine of tuberculosis patients at each concentration.

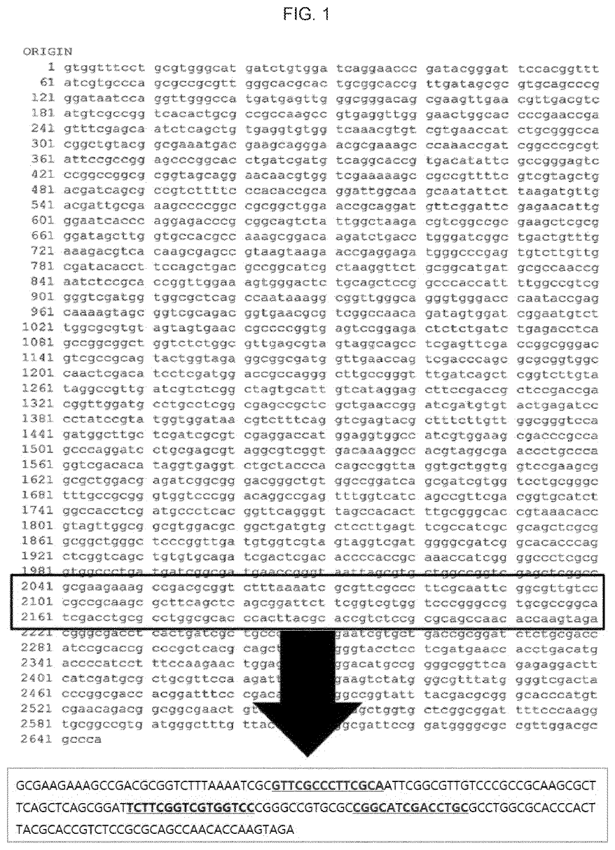

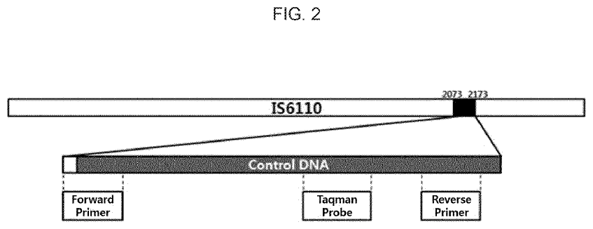

### DETAILED DESCRIPTION

The present invention is described in more detail by the following examples. These examples illustrate the present invention, and the scope of the present invention should not be limited thereby.

### Example 1. Preparation of Template DNA

The present inventors prepared the M. tuberculosis trDNA of SEQ ID NO: 2 identified in the urine sample of a tuberculosis patient as a template DNA.

Specifically, a template DNA was designed with a base sequence of 99 bp from the 2075^{th} base to the 2173^{rd} base in the M. tuberculosis IS6110 gene having the base sequence of SEQ ID NO: 1, and the template DNA designed for two-stranded base sequences complementary to each other so as to have a double linear structure was synthesized and prepared by commissioning an external organization (BIONEER Corporation).

The concentration of the synthesized template DNA was 6 μg/μl, and the synthesized template DNA was diluted decimally with 1×PBS (log 10 values were diluted to 6, 7, 8, 9, 10, and 11) and stored at −80° C. until use.

### Example 2. Design and Preparation of Primer and Probe

Based on a base sequence from the 1^{st} base to the 12^{th} base (base sequence from the 2073^{rd} base to the 2086^{th} base in the base sequence of SEQ ID NO: 1) in the base sequence of SEQ ID NO: 2 that is M. tuberculosis trDNA, and a base sequence from the 81^{st} base to the 95^{th} base in the base sequence of SEQ ID NO: 2, forward and reverse primers were synthesized as shown in Table 1 by selecting each base sequence such that Tm values were 52.5° C. to 56.3° C.

TABLE 1

| SEQ ID NO. | Forward Primer (forward) | SEQ ID NO. | Reverse Primer (reverse) |
|---|---|---|---|
| 3 | GTTCGCCCTTCGCA | 4 | GCAGGTCGATGCCG |

In addition, based on a base sequence from 54^{th} base to 69^{th} base in the base sequence of SEQ ID NO: 2, a probe, as shown in Table 2, was designed by selecting a base sequence such that the Tm value was 61.6° C. and an external organization (INTEGRATED DNA TECHNOLOGY (IDT, USA)) was commissioned to synthesize a reporter and a quencher which were designated FAM and BHQ1, respectively.

TABLE 2

| SEQ ID NO. | TaqMan Probe (forward) |
|---|---|
| 5 | tctt + Cg + Gtc + Gtg + Gtcc |

### Example 3. Gene Amplification Test by Real-Time PCR

After each template DNA (each template DNA sample whose log 10 values were diluted to 6, 7, 8, 9, and 10) prepared in Example, 1 and a DNA-free blank sample as control were each employed as a template and mixed using a primer set of SEQ ID NOS: 3 and 4 prepared in Example 2, a probe of SEQ ID NO: 5 and IQ supermix (Bio-Rad, USA), real-time PCR was performed using the C1000™ Thermal Cycler (manufactured by Bio-Rad Laboratories, Inc., USA) and the CFX96™ Real-Time System (manufactured by Bio-Rad Laboratories, Inc., USA).

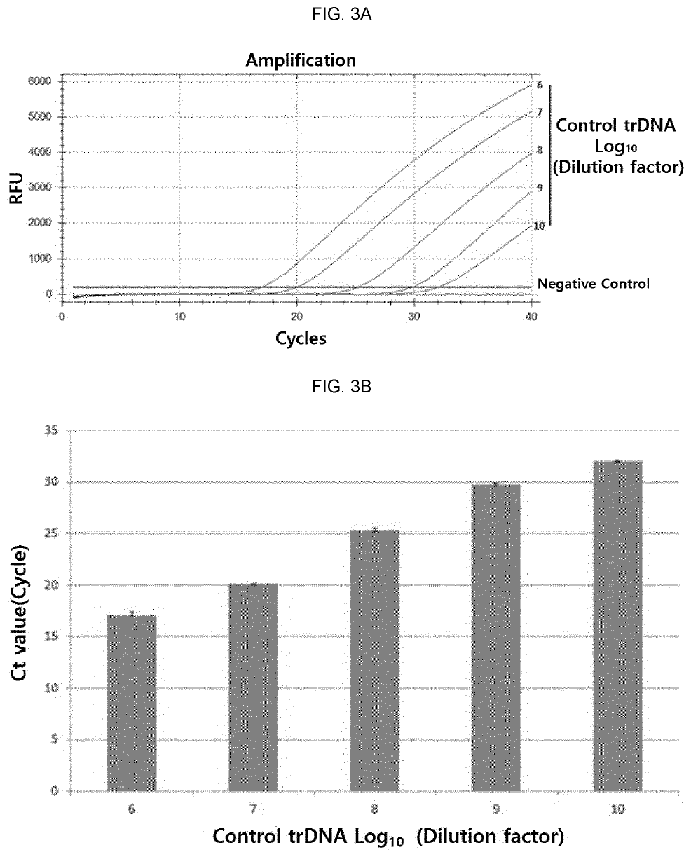

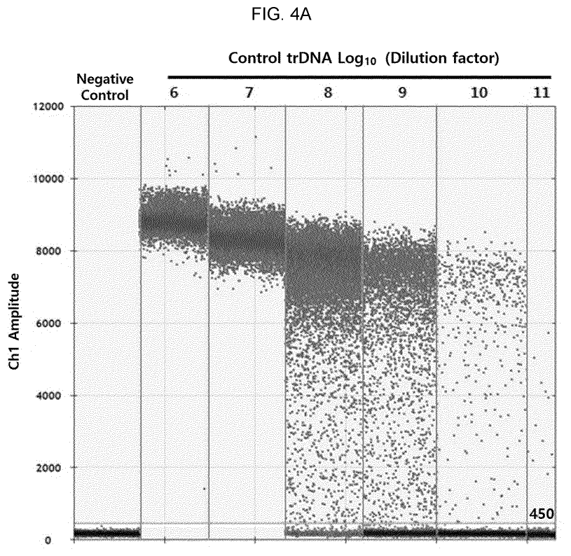

As reaction conditions, after denaturation at 95° C. for 3 minutes, fluorescence values were confirmed while performing 40 cycles of reactions at 95° C. for 10 seconds, at 55° C. for 10 seconds, and 72° C. for 30 seconds, and the results are shown in FIGS. 3A and 3B.

As illustrated in FIGS. 3A and 3B, it can be seen that the sensitivity for detecting a template DNA (M. tuberculosis trDNA) is high because all the template DNA is stably amplified until the template DNA is diluted to a low concentration (Log 10=10) when the primer and probe according to the present invention are used.

### Example 4. Gene Amplification Test by Droplet Digital PCR

After each template DNA (each template DNA sample whose Log 10 values were diluted to 6, 7, 8, 9, 10, and 11) prepared in Example, 1 and a DNA-free blank sample as control were each employed as a template and mixed using a primer set of SEQ ID NOS: 3 and 4 prepared in Example 2, a probe of SEQ ID NO: 5 and ddPCR supermix (No UTP) (Bio-Rad, USA) to produce a droplet by the QX200 Droplet Generator, a droplet digital polymerase chain reaction was performed using the C1000 Thermal Cycler (manufactured by Bio-Rad Laboratories, Inc., USA).

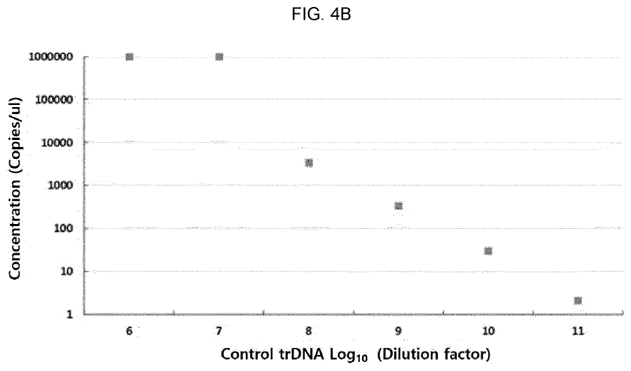

As reaction conditions, after denaturation at 95° C. for 10 minutes, 40 cycles of reactions at 94° C. for 30 seconds and at 60° C. for 1 minute were performed, and a reaction was performed at 98° C. for 10 minutes. The fluorescence values of the completely reacted droplet were read and analyzed using the QX200 Droplet Reader and Quanta Soft, and the results are illustrated in FIGS. 4A and 4B.

As illustrated in FIGS. 4A and 4B, it can be seen that the sensitivity for detecting a template DNA (M. tuberculosis trDNA) is high because all the template DNA is stably amplified until the template DNA is diluted to a low concentration (Log 10=11) when the primer and probe according to the present invention are used.

### Example 5. Gene Amplification Test Using Standard Strain (H37Rv)

A gene amplification test was performed using standard strain (H37Rv strain) DNA.

Specifically, H37Rv strain colonies cultured in an Ogawa medium at 37° C. for 1 month were collected by a loop and killed at 98° C. for 10 minutes. Then DNA was extracted using a Qiagen DNA extraction kit.

The extracted DNA concentration was 0.2 μg/μl, and H37Rv DNA was prepared by decimal dilution with 1×PBS (Log₁₀ values were diluted to 3, 4, 5, and 6).

<Real-Time PCR>

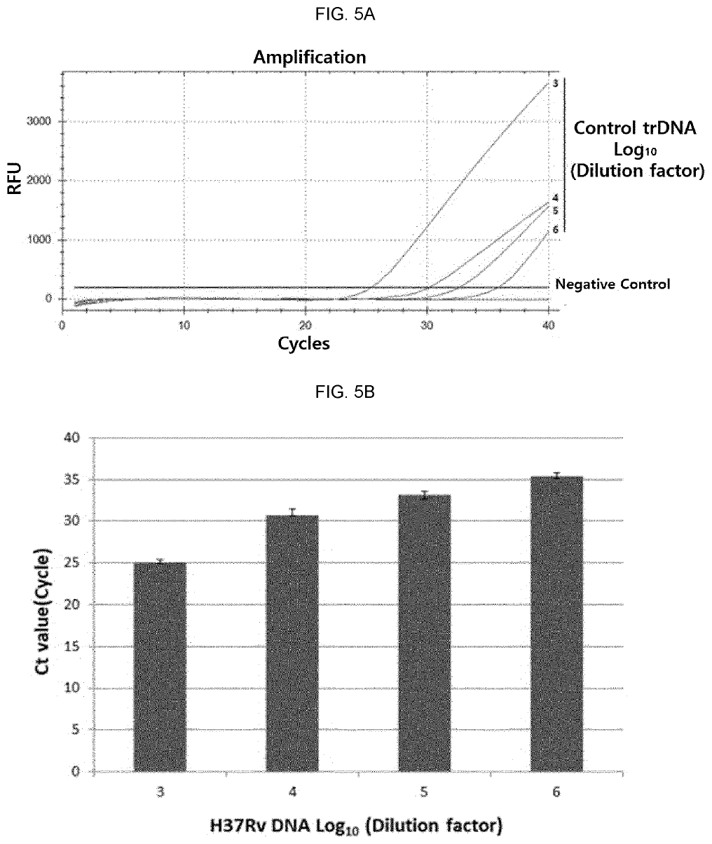

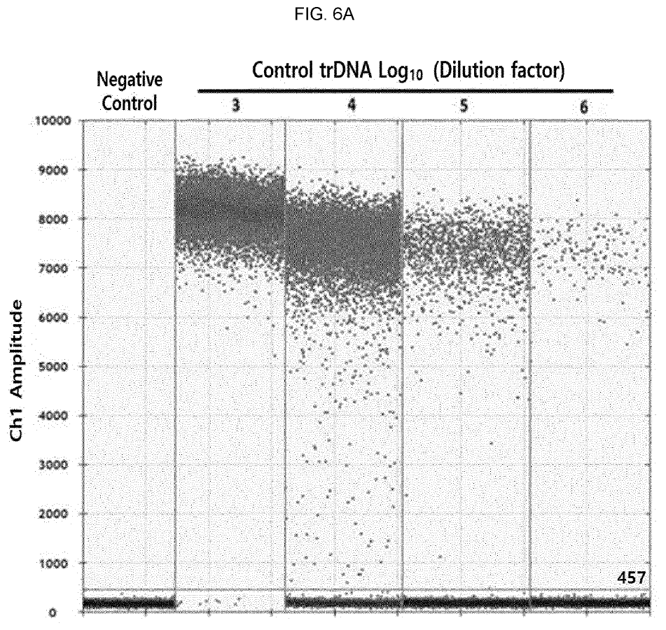

Real-time PCR was performed in the same manner as in Example 3, except that the H37Rv DNA prepared above was used as a template DNA, fluorescence values were confirmed in real-time, and the results are illustrated in FIGS. 5A and 5B.

As illustrated in FIGS. 5A and 5B, it can be seen that the sensitivity for detecting a DNA (M. tuberculosis trDNA) is high because all the template DNA is stably amplified until the standard strain H37Rv DNA is diluted to a low concentration (Log 10=6) when the primer and probe according to the present invention are used.

<Droplet Digital PCR>

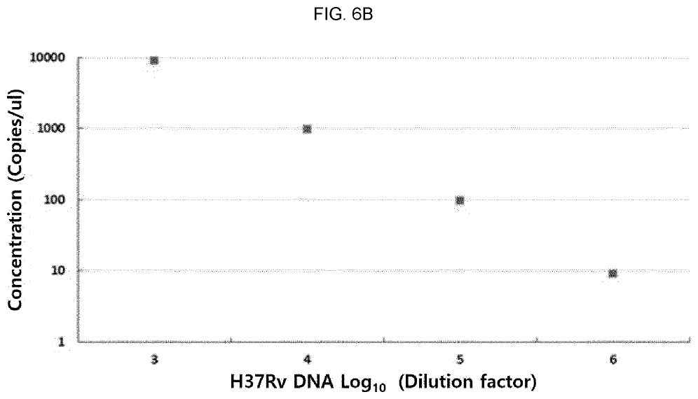

Droplet digital PCR was performed in the same manner as in Example 4, except that the H37Rv DNA prepared above was used as a template DNA, the fluorescence values of the completely reacted droplet were read and analyzed using the QX200 Droplet Reader and Quanta Soft, and the results are illustrated in FIGS. 6A and 6B.

As illustrated in FIGS. 6A and 6B, it can be seen that the sensitivity for detecting M. tuberculosis trDNA is high because all the template DNA is stably amplified until the template DNA is diluted to a low concentration (Log 10=6) when the primer and probe according to the present invention are used.

### Example 6. M. tuberculosis trDNA Detection (Gene Amplification) Test in Urine of Clinical Specimen (Tuberculosis Patient)

<Sample Collection and Storage>

2.5 ml of a cell-free DNA urine preserve (Streck) was added to 20 ml of urine collected at 8 am from 5 tuberculosis patients at Masan National Tuberculosis Hospital. The resulting mixture was stored in a refrigerator at −4° C. for up to 1 week.

M. tuberculosis trDNA was extracted from each urine sample using an Apintech MX-8 automatic nucleic acid extractor and an Apintech automatic nucleic acid extraction kit.

<Real-Time PCR>

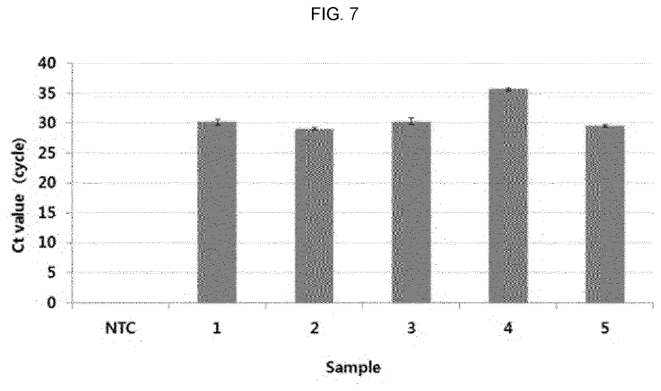

Real-time PCR was performed in the same manner as in Example 3, except that the M. tuberculosis trDNA prepared above was used as a template DNA, fluorescence values were confirmed in real-time, and the results are illustrated in FIG. 7.

As illustrated in FIG. 7, it can be seen that the sensitivity for detecting M. tuberculosis trDNA is high because all the template DNA is stably amplified even at a very low concentration of M. tuberculosis trDNA extracted from urine samples of 5 tuberculosis patients when the primer and probe according to the present invention are used.

<Droplet Digital PCR>

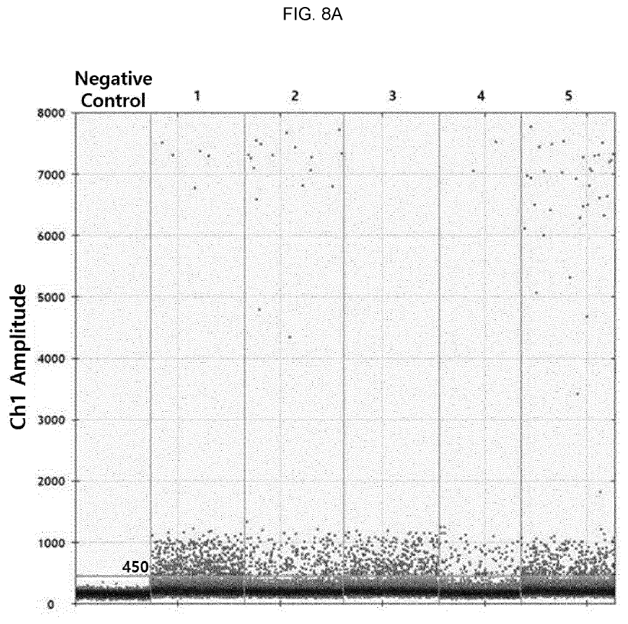

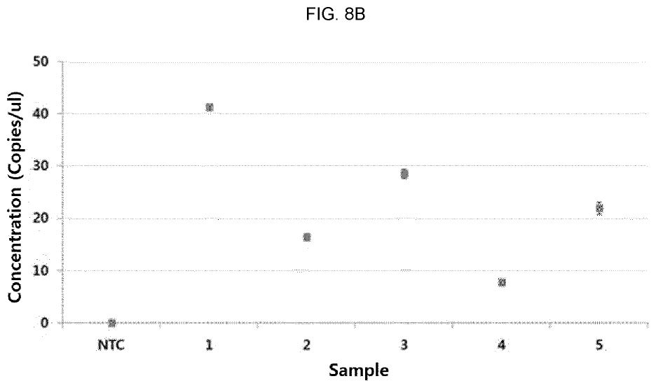

Droplet digital PCR was performed in the same manner as in Example 4, except that the M. tuberculosis trDNA prepared above was used as a template DNA, the fluorescence values of the completely reacted droplet were read and analyzed using the QX200 Droplet Reader and Quanta Soft, and the results are illustrated in FIGS. 8A and 8B.

As illustrated in FIGS. 8A and 8B, it can be seen that the sensitivity for detecting M. tuberculosis trDNA is high because all the template DNA is stably amplified even at a very low concentration of M. tuberculosis trDNA extracted from urine samples of 5 tuberculosis patients when the primer and probe according to the present invention are used.

1. A primer set for detecting M. tuberculosis trDNA in urine, comprising forward and reverse primers consisting of base sequences of 10 to 25 bp, which are identical to or complementary to a base sequence of M. tuberculosis trDNA of SEQ ID NO: 2.

2. The primer set of claim 1, wherein the primer consists of a base sequence of 14 to 20 bp.

3. The primer set of claim 1, wherein the primer consists of a base sequence of 16 bp.

4. The primer set of claim 1, wherein the forward primer consists of a base sequence of SEQ ID NO: 3 and the reverse primer consists of a base sequence of SEQ ID NO: 4.

5. A probe for detecting M. tuberculosis trDNA in urine consists of a base sequence of 10 to 25 bp, which is identical to or complementary to a base sequence of M. tuberculosis trDNA of SEQ ID NO: 2.

6. The probe of claim 5, wherein the 5′ end of the probe is labeled with a reporter, and the 3′ end of the probe is labeled with a quencher.

7. The probe of claim 6, wherein the reporter is anyone selected from the group consisting of 6-carboxyfluorescein (FAM), hexachloro-6-carboxyfluorescein (HEX), tetrachloro-6-carboxyfluorescein, and Cyanine-5 (Cy5), and the quencher is any one selected from the group consisting of 6-carboxytetramethyl-rhodamine (TAMRA) and black hole quencher-1,2,3 (BHQ-1,2,3).

8. The probe of claim 7, wherein the reporter is FAM, and the quencher is BHQ-1,2,3.

9. A kit for detecting M. tuberculosis trDNA in urine, comprising the primer set of claim 1.

10. The kit of claim 9, the kit further comprises the probe of claim 5.

11. The kit of claim 9, the kit further comprises an amplification buffer, dNTPs, a control, a detection reagent, and an instruction manual.

12.-15. (canceled)


SEQUENCE LISTING (5 sequences)

SEQ ID NO: 1 - DNA, 2645 bp, Mycobacterium tuberculosis
gtggtttcctgcgtgggcatgatctgtggatcaggaacccgatacgggattccacggtttatcgtgcccagcgccgcgttgggcacgcactgcggcaccgttgatagcgcgtgcagcccgggataatccaggttgggccatgatgagttgggcgggacagcgaagttgaacgttgacgtcatgtcgccggtcacactgcgccgccaagccgtgaggttgggaactggcaccccgaaccgagtttcgagcaatctcagctgtgaggtgtggtcaaacgtgtcgtgaaccatctgcgggccacggctgtacggcgaaatgacgaagcagggaacgcgaaagcccaaaccgatcggcccgcgtattccgccggagcccggcacctgatcgatgtcaggcaccgtgacatattcgccgggagtcccggccggcgcggtagcaggaacaacgtggtcgaaaaagccgccgttttcgtcgtagctgacgatcagcgccgtcttttcccacaccgcaggattggcaagcaatattcttaagatgttgacgattgcgaaagccccggccgcggctggaaccgcaggatgttcggattcgagaacattgggaatcacccaggagacccgcggcagtctattggctaagacgtcggccgcgaagctcgcgggatagcttggtgccacgccaaagcggacaagatctgacctgggatcggctgactgtttgaaagacgtcacaagcgagccgtaagtaagaaccgaggagatgggcccgagtgtcttgttgcgatacaccttccagctgacgccggcatcgctaaggttctgcggcatgatgcgccaaccgaatctccgcaccggttggaaagtgggactctgcagctccggcccaccattttggccgtcggggtcgatggtggcgctcagccaataaaggcggttgggcagggtgggacccaataccgagcaaaagtagcggtcgcagacggtgaacgcgtcggccaacagatagtggatcggaatgtcttggcgcgtgtagtagtgaaccgccccggtgagtccggagactctctgatctgagacctcagccggcggctggtctctggcgttgagcgtagtaggcagcctcgagttcgaccggcgggacgtcgccgcagtactggtagaggcggcgatggttgaaccagtcgacccagcgcgcggtggccaactcgacatcctcgatggaccgccagggcttgccgggtttgatcagctcggtcttgtataggccgttgatcgtctcggctagtgcattgtcataggagcttccgaccgctccgaccgacggttggatgcctgcctcggcgagccgctcgctgaaccggatcgatgtgtactgagatcccctatccgtatggtggataacgtctttcaggtcgagtacgctttcttgttggcgggtccagatggcttgctcgatcgcgtcgaggaccatggaggtggccatcgtggaagcgacccgccagcccaggatcctgcgagcgtaggcgtcggtgacaaaggccacgtaggcgaaccctgcccaggtcgacacataggtgaggtctgctacccacagccggttaggtgctggtggtccgaagcggcgctggacgagatcggcgggacgggctgtggccggatcagcgatcgtggtcctgcgggctttgccgcgggtggtcccggacaggccgagtttggtcatcagccgttcgacggtgcatctggccacctcgatgccctcacggttcagggttagccacactttgcgggcaccgtaaacaccgtagttggcggcgtggacgcggctgatgtgctccttgagttcgccatcgcgcagctcgcggcggctgggctcccggttgatgtggtcgtagtaggtcgatggggcgatcggcacacccagctcggtcagctgtgtgcagatcgactcgacaccccaccgcaaaccatcggggccctcgcggtggccctgatgatcggcgatgaaccgggtaattagcgtgctggccggtcgagctcggccgcgaagaaagccgacgcggtctttaaaatcgcgttcgcccttcgcaattcggcgttgtcccgccgcaagcgcttcagctcagcggattcttcggtcgtggtcccgggccgtgcgccggcatcgacctgcgcctggcgcacccacttacgcaccgtctccgcgcagccaacaccaagtagacgggcgacctcactgatcgctgcccactccgaatcgtgctgaccgcggatctctgcgaccatccgcaccgcccgctcacgcagctccggcgggtacctcctcgatgaaccacctgacatgaccccatcctttccaagaactggagtctccggacatgccggggcggttcagagaggacttcatcgatgcgctgcgttccaagattggcgagaagtctatgggcgtttatggggtcgactacccggcgaccacggatttcccgacagcgatggccggtatttacgacgcgggcacccatgtcgaacagacggcggcgaactgtccccaaagcaagctggtgctcggcggattttcccaaggtgcggccgtgatgggctttgttaccgcggcggcgattccggatggggcgccgttggacgcgccca

SEQ ID NO: 2 - DNA, 99 bp, Mycobacterium tuberculosis
tcgcccttcgcaattcggcgttgtcccgccgcaagcgcttcagctcagcggattcttcggtcgtggtcccgggccgtgcgccggcatcgacctgcgcct

SEQ ID NO: 3 - DNA, 14 bp, Artificial Sequence
  Forward Primer
gttcgcccttcgca

SEQ ID NO: 4 - DNA, 14 bp, Artificial Sequence
  Reverse Primer
gcaggtcgatgccg

SEQ ID NO: 5 - DNA, 16 bp, Artificial Sequence
  TaqMan Probe
tcttcggtcgtggtcc